(19) United States
(12) Patent Application Publication
(10) Pub. No.: US 2020/0071646 A1
(43) Pub. Date: Mar. 5, 2020

(54) Module For Photobioreactor And Associated Photobioreactor

(71) Applicants: Centre National de la Recherche Scientifique (CNRS), Paris (FR); Universite De Nantes, Nantes (FR); Ecole Nationale Superieure De Chimie De Clermont-Ferrand, Aubiere (FR)

(72) Inventors: Jérémy Pruvost, Saint-Brevin Les Pins (FR); Jack Legrand, Saint Nazaire (FR); Jean-Francois Cornet, Durtol (FR)

(73) Assignees: Centre National de la Recherche Scientifique (CNRS), Paris (FR); Universite De Nantes, Nantes (FR); Ecole Nationale Superieure De Chimie De Clermont-Ferrand, Aubiere (FR)

(21) Appl. No.: 16/463,893

(22) PCT Filed: Nov. 24, 2017
(86) PCT No.: PCT/EP2017/080380

§ 371 Date: May 24, 2019

(30) Foreign Application Priority Data
Nov. 25, 2016 (FR) 1661496

(51) Int. Cl. C12M 1/00; C12M 1/12; C12M 3/00

(52) U.S. Cl. CPC C12M 21/02; C12M 23/04; C12M 23/44; C12M 29/24; C12M 29/06; C12M 31/08; C12M 31/10; C12M 23/58

(57) ABSTRACT

The present invention concerns a module for a photobioreactor suitable for being assembled to an identical adjacent module, comprising: —a panel having a first face and a second face, opposite the first face, the first face delimiting, with the second face of the adjacent panel, a cavity suitable for containing a culture medium when the modules are assembled, the cavity (14) having a thickness of between 1 millimetre and 2 centimetres, and —a gas injection device, capable of injecting gas bubbles into the culture medium contained in the cavity, such that the bubbles rise along the faces of the panels.

### FIELD OF THE INVENTION

This invention relates to a system for producing photosynthetic microorganisms suspended in water.

### STATE OF THE ART

A photobioreactor is a system that produces photosynthetic microorganisms suspended in water, such as multicellular plants, small plants such as gametophytes, photosynthetic bacteria, cyanobacteria, eukaryotic microalgae, isolated macroalgal cells and moss protonemata.

This production involves culture, most often clonal, in an aqueous medium under illumination. Amplification to industrial volumes up to hundreds of cubic meters is carried out in successive steps where the volume of one step is used to inoculate the next volume. To harvest the microbial population and ensure biomass production, the volume of each step can be partially renewed daily (continuous culture) or completely changed (batch culture). These steps correspond to photobioreactors of increasing volume.

Photosynthetic microorganisms produce biomass only when they receive a quantity of light energy, called irradiance, greater than a minimum irradiance Gc. However, since an algae culture is an absorbent medium that attenuates light, the light decreases with the thickness of the culture according to an exponential law.

To increase the biomass productivity of culture systems, it is advisable to increase the supply of light energy and/or decrease the culture volume for a given illumination surface (i.e. increase the specific surface illuminated). Thus, productivity in terms of culture volume (i.e. volume productivity) is usually distinguished from productivity in terms of illuminated surface (i.e. surface productivity).

In order to be able to change the volume of the photobioreactor during the steps described above, and to standardize the irradiance received by the microorganisms, the Toshihiko Kondo document “Efficient hydrogen production using a multi-layered photobioreactor and a photosynthetic bacterium mutant with reduced pigment” proposed multi-layered photobioreactors which dilute strong incident light and diffuse it uniformly in the reactor. Layers can be gradually added to the photobioreactor to allow the volume of the photobioreactor to be changed.

However, systems designed on this principle face a major limitation, namely the tendency to form deposits, called biofilms, on the illuminating walls, which over time decreases the light intensity in the reactor volume and therefore production efficiency. This phenomenon is all the greater when the culture thickness (i.e. the distance between light plates) is small. The confinement of the medium indeed increases, as well as the biomass concentration, due to the increase in the specific illuminating surface area, which leads to higher volume productivity.

To avoid biofilm formation, it has been proposed to insert mechanical cleaning devices into the cavity receiving the culture medium, but such cleaning devices are not compatible with thin cavities. In practice, the culture thickness must therefore be at least a few centimeters, typically greater than 1 cm, which does not allow high volume productivity to be obtained.

In the end, these systems, designed on the principle of submerged illuminated plates, have low volume productivity and a strong tendency to foul.

In terms of technologies allowing high volume productivity, one example is the document FR2950899, which describes a photobioreactor comprising an illumination panel inclined by an average slope in a direction of inclination over which a solution flows. This makes it possible to obtain a minimum thickness of a few millimeters and therefore high volume productivity. Thus, the illumination panel is not in contact with the culture, which prevents biofilms. Such a solution is, however, incompatible with a modular photobioreactor with internal illumination.

### DISCLOSURE OF THE INVENTION

One aim of the invention is to propose a solution that will allow the volume of the photobioreactor to be changed while maintaining an intensified and efficient volume production of biomass.

This aim is achieved in the context of the present invention by means of a photobioreactor module adapted to be assembled with an identical adjacent module, comprising:
a panel having a first side and a second side, opposite the first side, the first side delimiting, with the second side of the adjacent panel, a cavity suitable for containing a culture medium when the modules are assembled, the cavity having a thickness comprised between 1 millimeter and 2 centimeters, and
a gas injection device, suitable for injecting gas bubbles into the culture medium contained in the cavity, so that the bubbles rise up the sides of the panels.

The culture thickness, defined by the spaces between the plates, is very low, which allows high volume productivities. Biofilm formation is prevented by effective bubbling between the two plates. Indeed, due to the thinness of the cavity, the gas bubbles rising up the sides of the panels mechanically clean said panels as they pass. The injection of gas, necessary for the culture of microorganisms (supply of nutrients), also helps to promote the movement of the culture medium, which makes it possible to standardize the irradiation of microorganisms present in the cavity.

The module is also designed to guarantee constant volume and surface productivities. The modular nature of the photobioreactor indeed makes it possible to increase production capacity in a simple and linear way, by adding modules that maintain optimal exposure of the microorganisms to light. The modules can be added to the photobioreactor to be able to change the volume of the photobioreactor from a few liters to several m³. Due to the thinness of the cavity and the illumination on both sides (the culture thickness is therefore equal to half the cavity thickness), such a module makes it possible to manufacture a photobioreactor with high volume productivity, regardless of the number of modules assembled.

FIG. 8 shows the volume productivity per day Pv (log plot) as a function of culture thickness L, for a luminous flux (PFD) of 800 μmol·m−1·s−1 (solid line), and for a luminous flux (PFD) of 200 μmol·m−1·s−1 (dotted line). This graph shows that the invention makes it possible to produce more than 10 times more than the prior art.

The invention is advantageously completed by the following features, taken individually or in any one of their technically possible combinations.
the module comprises an illumination device, suitable for illuminating the culture medium contained in the cavity, the illumination device comprising a woven optical fiber web or a diffusing glass plate.
the panel comprises two transparent plates between which the illumination device extends;
each transparent plate is made of poly(methyl methacrylate), glass or any other transparent material;
the gas injection device comprises gas injection channels cut or inserted into one of the transparent plates;
the panel comprises an opening allowing fluid communication between cavities when several modules are assembled;
the first and/or second side of the panel have/has a recess to form the cavity;
the module comprises a gasket arranged to create a tight contact between the modules;
the module comprises a frame extending along the edges of the panel to stiffen the panel.

The invention also relates to a photobioreactor, comprising an assembly of several modules as described above, each module defining, with an adjacent module, a cavity suitable for containing the culture medium.

The photobioreactor comprises an assembly of several modules and a control unit configured to control the injection of gas bubbles into the culture medium contained in the cavity, alternately over a first section of the width of the cavity, then over a second section of the width of the cavity.

The photobioreactor comprises of a frame with guide rails on which the modules are supported so as to keep the modules in position relative to each other.

The photobioreactor comprises a device for pressing the assembly to keep the modules in tight contact with each other.

The pressing device comprises a first end plate and a second end plate, between which the modules are arranged, and an actuator suitable for exerting pressure on the second plate to bring the second plate closer to the first plate.

The photobioreactor comprises a device for controlling the temperature of the culture medium.

The modules include an instrumented module, the instrumented module comprising one or more sensors in contact with the culture medium.

### DESCRIPTION OF THE FIGURES

Other objectives, features and advantages will emerge from the following detailed description with reference to the drawings given by way of non-limiting illustration, wherein:

FIG. 1 represents a photobioreactor in accordance with one embodiment of the invention;

FIG. 2 represents a module in accordance with one embodiment of the invention;

FIG. 3 is a cross-sectional view of a module in accordance with one embodiment of the invention;

FIG. 4 is a cross-sectional view of a module assembly;

FIG. 5 is a cross-sectional view of a module in accordance with one embodiment of the invention;

FIG. 6 is an exploded view of a photobioreactor in accordance with one embodiment of the invention;

FIG. 7 is a cross-sectional view of a module assembly with an instrumentation module;

FIG. 8 shows the volume productivity per day (plotted in log) as a function of culture thickness.

### DETAILED DESCRIPTION OF THE INVENTION

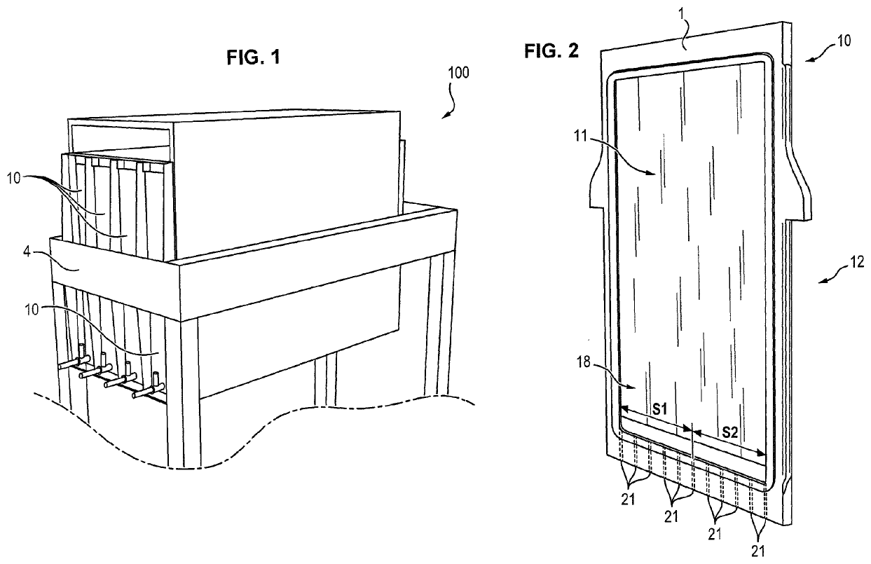

With reference to FIG. 1, a photobioreactor 100 comprises of a frame 4 and a series of modules 1.

With reference to FIG. 4, each photobioreactor module 10 is adapted to be assembled with an identical adjacent module so as to define, with the latter, a cavity 14 having an opening 8 provided in the upper part and suitable for containing a culture medium. Each module 10 comprises a panel 1, a gas injection device 21.

The distance between two panels 1 defines the thickness of the cavity 14, while the width of the panel 1 defines the width of the cavity 14, and the height of the panel 1 the height of the cavity 14.

With reference to FIGS. 2, 3 and 4, each panel 1 has a first side 11 and a second side 12, opposite the first side 11. The first side 11 delimits, with the second side 12 of the adjacent panel 1, a cavity 14 suitable for containing culture medium when the modules 10 are assembled. The cavity 14 has a thickness e comprised between 1 millimeter and 2 centimeters, preferably comprised between 1 millimeter and 9 millimeters, for example 6 millimeters.

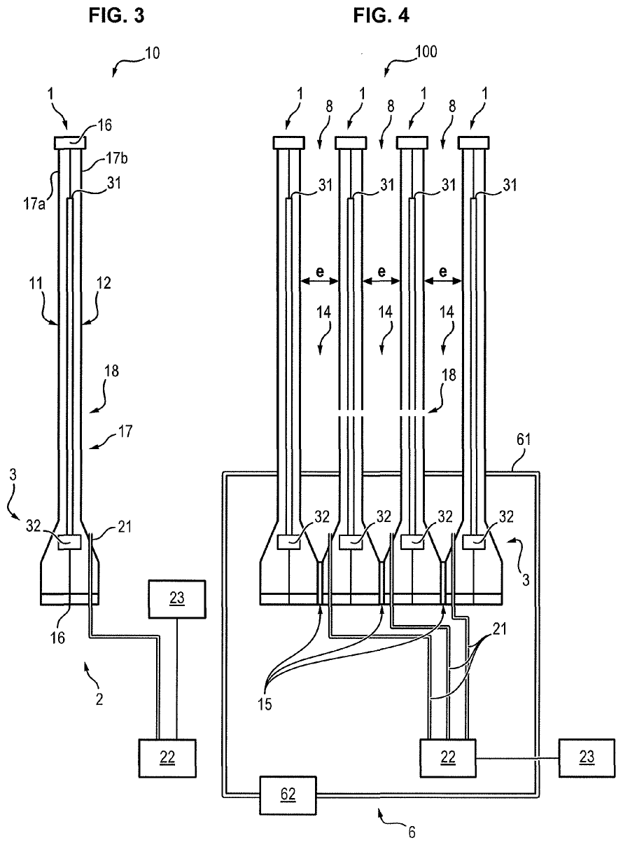

With reference to FIG. 3, each panel 1 comprises two transparent plates 17a, 17b. The transparent plates 17a, 17b are made with materials such as glass or rigid plastics (for example methyl methacrylate (PMMA), or polycarbonate) or with flexible plastic films (for example polyethylene, polyurethane, or vinyl chloride—PVC). Each module 10 comprises a frame 16 extending along the edges of the panel to stiffen the panel. The first side 11 and the second side 12 of the panel 1 have a recess 17 to form the cavity 14. More precisely, each of the plates 17a and 17b is formed into a single piece of material, the recess 17 being formed in the plate 17a and/or in the plate 17b.

With reference to FIG. 4, each module 10 comprises a gasket 15 arranged to create a tight contact between the modules 10 when assembled.

With reference to FIG. 3, the gas injection device 2 is suitable for injecting gas bubbles into the culture medium contained in the cavity 14, so that the bubbles rise up the sides 11 and 12 of the panels 1. The injected gas contains carbon dioxide and is typically air or gas from a reaction and collected coming out of the factory.

The gas injection device 2 comprises gas injection channels 21 distributed over the width of the cavity 14 in the lower part of the panels 1. The gas injection channels 21 are cut into one of the transparent plates 17a, 17b of each module 10 or arranged between the two transparent plates 17a, 17b. The gas injection device 2 typically comprises a tank 22 containing the gas to be injected under pressure, the tank having an outlet in communication with the gas injection channels 21.

The gas injection device 2 injects the gas loaded with carbon dioxide required by the microorganisms via the gas injection channels 21 into the cavity 14. The injected gas forms bubbles that rise up the sides 11, 12 of the panels 1. As they rise, the bubbles are loaded with photosynthetic oxygen generated by the microorganisms. The gas loaded with photosynthetic oxygen generated by the microorganisms is evacuated through an opening 8 provided in the upper part of the panels.

The gas injection device 2 renews the carbon dioxide consumed by the microorganisms and eliminates the photosynthetic oxygen generated by the microorganisms. Indeed, a concentration too high in oxygen and too low in carbon dioxide inhibits the photosynthetic reaction.

By rising up the sides of the panels 11 and 12, the bubbles mechanically clean the inner wall of the cavity 14, preventing the development of a biofilm that would reduce the light intensity in the reactor volume.

The injection of gas also promotes the movement of the culture medium, in order to improve the uniformity of the irradiation of the microorganisms.

The gas injection device 2 is advantageously adapted to inject gas in a pulsed and sequential manner in order to increase the shear effect on the forming biofilm.

A control unit 23 configured to control the injection of gas bubbles into the culture medium contained in the cavity 14, controls the injection of the bubbles alternately the gas injection channels 21 distributed over a first section S1 of the width of the cavity 14, then the gas injection channels 21 distributed over a second section S2 of the width of the cavity 14.

The control unit 23 can also be configured to control alternately the gas injection channels 21 distributed over the first section S1 of a first group of cavities 14, then the gas injection channels 21 distributed over the second section S2 of a second group of cavities 14.

The control unit 23 can also be configured to control alternately the gas injection channels 21 distributed over the first section S1 of a first group of cavities 14 and the gas injection channels 21 distributed over the second section S2 of a second group of cavities 14, then the gas injection channels 21 distributed over the second section S2 of the first group of cavities 14 and the gas injection channels 21 distributed over the first section S1 of the second group of cavities 14.

The first section S1 of the width of the cavity 14 is typically complementary to the second section S2 of the width of the cavity 14.

To that end, the control unit 23 controls the injection of bubbles according to the following sequence:

injection of bubbles through the gas injection channels 21 distributed over a first section S1 of the width of the cavity 14,

injection of bubbles through the gas injection channels 21 distributed over a second section S2 of the width of the cavity 14.

The injection sequence may further include a step of injecting bubbles simultaneously through the gas injection channels 21 distributed over a first section S1 of the width of the cavity 14, and through those distributed over the second section S2 of the width of the cavity 14.

The inventors have shown that injecting gas bubbles alternately on one section S1 of the width of the cavity 14 and then on the other section S2 of the width of the cavity 14 substantially improved the cleaning efficiency.

Indeed, the injection of bubbles entrains the liquid upward. This upward movement is compensated by a downward movement of liquid. However, this downward movement of liquid disrupts the shear effect of the bubbles.

When gas bubbles are injected over a section of the width of the cavity 14 only, the bubbles entrain the liquid upward over said section of the width of the cavity 14 and the liquid descends to the complementary section of the width of the cavity 14 forming a cyclic movement. The downward movement of liquid thus does not disturb the shear effect of the bubbles. The cleaning of the complementary section of the width of the cavity 14 is then carried out by injecting gas bubbles into the complementary section of the width of the cavity 14. Thus, the cavity 14 is finally cleaned over its entire width.

Furthermore, the gas injection device 2 is advantageously adapted to form bubbles having a size of the same order of magnitude as the thickness e to increase the shear effect on the forming biofilm.

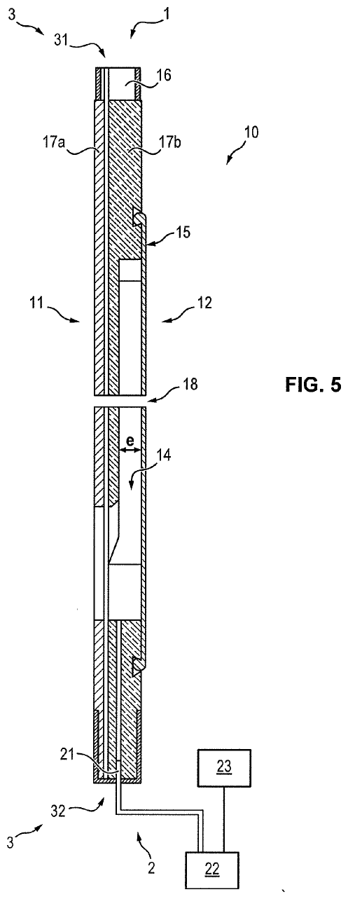

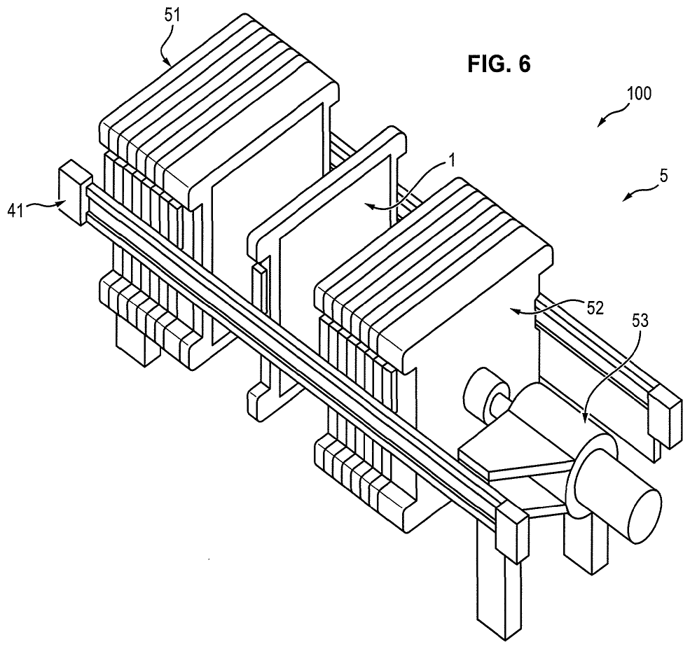

With reference to FIG. 5, each module 10 comprises an illumination device 3, suitable for illuminating the culture medium contained in the cavity 14, the illumination device 3 comprising a light source 32 and an optical diffusion device 31.

The light source 32 is typically a light-emitting diode. It typically emits white light in a spectral range adapted to improve photosynthetic conversion.

The light source 32 can be common to all the modules 10. In particular, the light source 32 can be sunlight. In this case, the light source 32 comprises a system for capturing the solar flux and transmitting the flux to the optical diffusion devices 31.

The optical diffusion device 31 extends between the two transparent plates 17a, 17b. The internal illumination allows total absorption of emitted flux, control of light attenuation conditions and therefore of biological conversion, and low energy consumption.

The optical diffusion device 31 is typically a side-diffusion device such as a laterally diffused woven optical fiber web or a diffusing glass plate, or a thin polymer sheet in which strips of light-emitting diodes (LEDs) are integrated.

With reference to FIG. 5, the panel 1 comprises an opening 18 allowing fluid communication between cavities 14 when several modules 10 are assembled. Thus, the modules 10 can be assembled together so that the cavities 14 are in fluid communication.

With reference to FIG. 6, the frame 4 typically comprises guide rails 41 on which the modules 10 are supported so as to keep the modules 10 in position relative to each other. The photobioreactor 100 further comprises a device 5 for pressing the assembly to keep the modules 10 in tight contact with each other. The pressing device 5 comprises a first end plate 51 and a second end plate 52, between which the modules 10 are arranged, and an actuator 53 suitable for exerting pressure on the second plate 52 to bring the second plate 52 closer to the first plate 51. The pressure exerted ensures watertightness between the different modules 10. The actuator 53 is typically a hydraulic jack driven by a pump (centrifugal or diaphragm for example).

With reference to FIG. 4, the photobioreactor 100 further comprises a device 6 for controlling the temperature of the culture medium, to maintain the culture temperatures at an optimal temperature in the photobioreactor. Indeed, a dense culture absorbs infrared radiation, which can lead to temperatures lethal to the microorganisms if the excess heat is not removed.

To that end, the temperature control device 6 comprises, for example, a flow circuit comprising a circulation tube 61 for a cooling liquid and a cooling module 62, the circulation tube 61 extending through the cooling module 62. The circulation tube 61 extends for example through the modules 10 or through the plates 17a, 7b. The coolant can be water, for example.

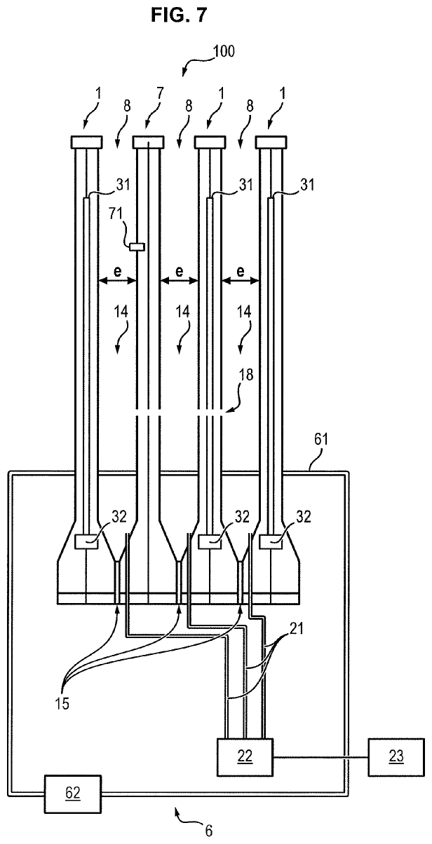

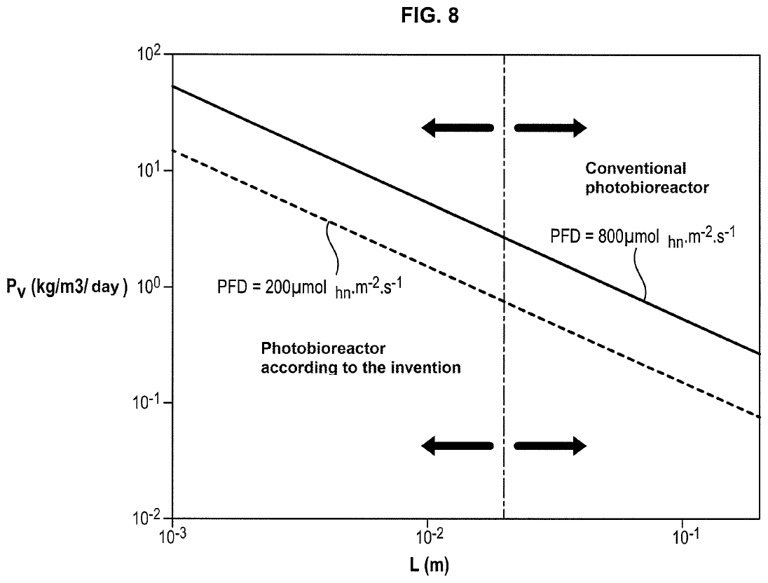

With reference to FIG. 7, the photobioreactor 100 further comprises at least one instrumented module 7. The instrumented module 7 is similar to the other modules 10 and comprises one or more sensors 71, such as for example a temperature sensor, or an oxygen concentration sensor, in contact with the culture medium. The information from these sensors is used to monitor conditions in the cavity—particularly temperature, illumination, and solution composition—in order to optimize the productivity of the culture.

1. Photobioreactor module adapted to be assembled with an identical adjacent module, comprising:
a panel having a first side and a second side, opposite the first side, the first side delimiting, with the second side of the adjacent panel, a cavity suitable for containing a culture medium when the modules are assembled, the cavity having a thickness comprised between 1 millimeter and 2 centimeters, and
a gas injection device, suitable for injecting gas bubbles into the culture medium contained in the cavity, so that the bubbles rise up the sides of the panels.

2. Module according to claim 1, comprising an illumination device, suitable for illuminating the culture medium contained in the cavity, the illumination device comprising a woven optical fiber web or a diffuser plate or a plate into which light-emitting diode type light sources are integrated.

3. Module according to claim 2, wherein the panel comprises two transparent plates between which the illumination device extends.

4. Module according to claim 3, wherein each transparent plate is made of poly(methyl methacrylate), glass or any other transparent material.

5. Module according to claim 3, wherein the gas injection device comprises gas injection channels cut or inserted into one of the transparent plates.

6. Module according to claim 1, wherein the panel comprises an opening allowing fluid communication between cavities when several modules are assembled.

7. Module according to claim 1, wherein the first and/or the second side of the panel have/has a recess to form the cavity.

8. Module according to claim 1, comprising a gasket arranged to create a tight contact between the modules.

9. Module according to claim 1, comprising a frame extending along the edges of the panel to stiffen the panel.

10. Photobioreactor, comprising an assembly of several modules according to claim 1, each module defining, with an adjacent module, a cavity suitable for containing the culture medium.

11. Photobioreactor, comprising an assembly of several modules according to claim 10, further comprising a control unit configured to control the injection of gas bubbles into the culture medium contained in the cavities, alternately over a first section of the width of the cavity, and then over a second section. of the width of the vacity.

12. Photobioreactor according to claim 10, comprising a frame comprising guide rails on which the modules are supported so as to keep the modules in position relative to each other.

13. Photobioreactor according to claim 10, comprising a device for pressing the assembly to keep the modules in tight contact with each other.

14. Photobioreactor according to claim 13, wherein the pressing device comprises a first end plate and a second end plate, between which the modules are arranged, and an actuator suitable for exerting pressure on the second plate to bring the second plate closer to the first plate.

15. Photobioreactor, according to claim 10, comprising a device for controlling the temperature of the culture medium.

16. Photobioreactor, according to claim 10, wherein the modules include an instrumented module, the instrumented module comprising one or more sensors in contact with the culture medium.
